(19) United States
(12) Patent Application Publication
(10) Pub. No.: US 2026/0334975 A1
(43) Pub. Date: Oct. 29, 2026

(54) IMAGING SYSTEM WITH NEEDLE ALIGNED TO FIELD OF VIEW

(71) Applicant: INTUITIVE SURGICAL OPERATIONS, INC., Sunnyvale, CA (US)

(72) Inventors: Lucas S. Gordon, Sunnyvale, CA (US); Samuel Raybin, San Jose, CA (US); Randall L. Schlesinger, San Mateo, CA (US); Serena H. Wong, Los Altos, CA (US)

(21) Appl. No.: 19/110,701

(22) PCT Filed: Sep. 11, 2023
(86) PCT No.: PCT/US2023/032385

§ 371 Date: Mar. 11, 2025

Related U.S. Application Data
(60) Provisional application No. 63/406,181, filed on Sep. 13, 2022.

(51) Int. Cl. A61B 17/34; A61B 1/018; A61B 10/04; A61B 17/00; A61M 25/00

(52) U.S. Cl. CPC A61B 17/3478; A61B 1/018; A61B 2010/045; A61B 2017/0034; A61B 2017/00867; A61B 2017/3413; A61M 2025/0092

(57) ABSTRACT

An apparatus may comprise an imaging probe including a channel extending through the imaging probe and terminating at an opening and an imaging device configured to generate image data having a field of view. The apparatus may also comprise a tool configured to slidably extend within the channel. A portion of the tool may be flexible and may bend away from the imaging device when the portion of the tool is extended distally of the opening.

### CROSS-REFERENCED APPLICATIONS

This application claims priority to and benefit of U.S. Provisional Application No. 63/406,181 filed Sep. 13, 2022 and entitled “Imaging System with Needle Aligned to Field of View,” which is incorporated by reference herein in its entirety.

### FIELD

The present disclosure is directed to systems and methods for aligning a tool within a field of view of an imaging system.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Medical tools may be inserted into anatomic passageways and navigated toward a region of interest within a patient anatomy. Navigation may be assisted using optical or ultrasound images of the anatomic passageways and surrounding anatomy, obtained intra-operatively. Intra-operative imaging of a tool by an imaging probe or catheter through which the tool is inserted may provide improved navigational guidance and confirmation of engagement of the tool with the target tissue. Improved systems and methods are needed to align a tool in a field of view of an imaging probe through which the tool is inserted to improve imaging of the tool and procedures performed with the tool.

### SUMMARY

Consistent with some examples, an apparatus may comprise an imaging probe including a channel extending through the imaging probe and terminating at an opening and an imaging device configured to generate image data having a field of view. The apparatus may also comprise a tool configured to slidably extend within the channel. A portion of the tool may be flexible and may bend away from the imaging device when the portion of the tool is extended distally of the opening.

In some examples, a system may comprise an imaging probe including a channel extending through the imaging probe and terminating at an opening and an imaging device configured to generate image data having a field of view. The system may also comprise a tool configured to slidably extend within the channel. A portion of the tool may be flexible and may bend away from the imaging device when the portion of the tool is extended distally of the opening. The system may also comprise a control system comprising one or more processors configured to deploy the portion of the tool distally of the opening and into the field of view and receive the image data including the portion of the tool in the field of view.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates an imaging probe with a tool, according to some examples.

FIG. 2 illustrates the imaging probe of FIG. 1 with an image of a field of view including an image of the tool, according to some examples.

FIG. 3 illustrates a tool including a plurality of perforations that promote preferential bending, according to some examples.

FIG. 4 illustrates an imaging probe with a tool, according to some examples.

FIG. 5 illustrates an imaging probe with a tool, according to some examples.

FIG. 6A-6E illustrate cross sectional views of a working channel of an imaging probe and a tool, according to various examples.

FIG. 7 illustrates a cross sectional view of a working channel of an imaging probe and a tool, according to some examples.

FIG. 8A illustrates an imaging probe with a tool rotationally constrained relative to the working channel, according to some examples.

FIG. 8B illustrates a cross sectional view of a working channel of an imaging probe and a tool, according to some examples.

FIG. 9 illustrates a tool with a flexible, bend portion, according to some examples.

FIG. 10 is a flowchart illustrating a medical imaging procedure using an imaging probe and a tool.

FIG. 11 illustrates a simplified diagram of a robot-assisted medical system according to some examples of the present disclosure.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

The systems and techniques disclosed in this document may be used to enhance minimally invasive procedures that provide intra-operative visualization using, for example, ultrasound imaging. Imaging data from an imaging probe may be utilized to verify real-time accurate placement of a treatment or diagnostic tool within an anatomical target during a medical procedure. In some examples, an imaging probe includes a channel through which the tool is inserted. The size of an imaging probe may be minimized by the tool having a bent shape upon exiting the imaging probe, thereby causing the distal end of the tool to be within the field of view of the imaging probe. In some examples, the tool may also or alternatively be constrained to prevent rotation relative to the imaging probe so that the tool bends into the field of view. Although described in the context of an ultrasound imaging probe, it is contemplated that the systems and methods described herein may be applied to other imaging modalities without departing from the scope of the present disclosure. For example, an imaging probe including a camera may be used to provide direct visual guidance of a tool as the tool is delivered via a flexible elongate device (e.g., a catheter or imaging probe) into a target. In another example, the imaging probe may be a fluoroscopic imaging device, or some other type of imaging device.

The systems and techniques described in this document may be used in a variety of medical procedures that may improve accuracy and outcomes through use of intra-operative imaging. For example, intra-operative imaging may be used to biopsy lesions or other tissue to, for example, evaluate the presence or extent of diseases such as cancer or surveil transplanted organs. As another example, intra-operative imaging may be used in cancer staging to determine via biopsy whether the disease has spread to lymph nodes. The medical procedure may be performed using hand-held or otherwise manually controlled imaging probes and tools (e.g., a bronchoscope). In other examples, the described imaging probes and tools many be manipulated with a robot-assisted medical system.

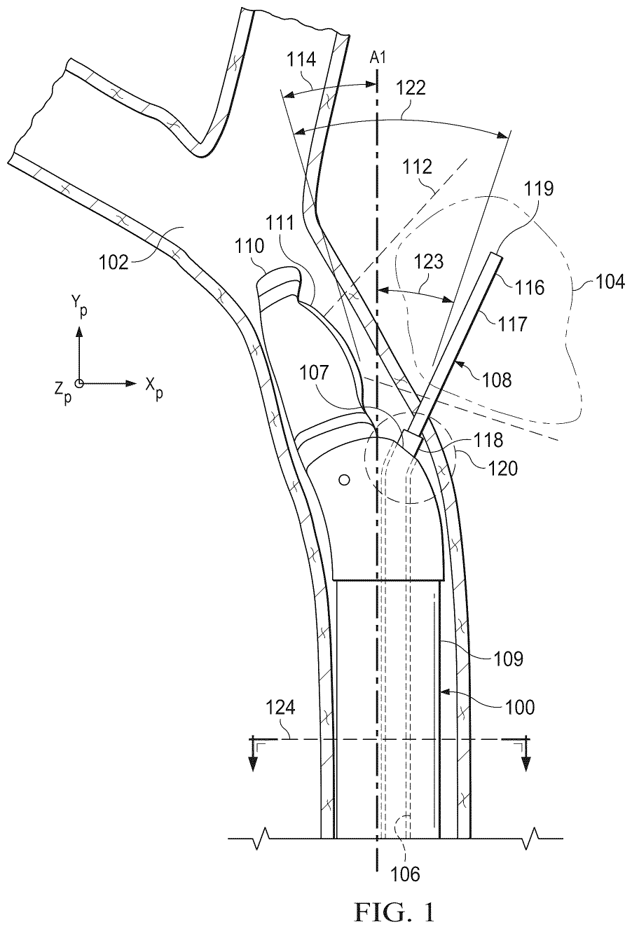

FIG. 1 illustrates a partial cross-sectional view of an imaging probe 100 extending within a passageway 102 of a patient anatomy near a target tissue 104 which may be the subject of imaging by the imaging probe 100. A frame of reference for the imaging probe 100 may have a coordinate system Xₚ, Yₚ, Zₚ. In some examples, the passageway 102 may be an airway of a lung and the target tissue 104 may be a lymph node, a suspected tumor, a nodule, or a lesion to be imaged by endobronchial ultrasound imaging during a biopsy procedure.

The imaging probe 100 may include at least one channel 106 through which tools, devices, or other elongated instruments may extend. In the illustrated example, a tool system 108 may slidably extend through the channel 106, out an opening or aperture 107 at a distal end of the channel 106, and into the target tissue 104. In some examples, the imaging probe 100 may include a flexible shaft 109 and an imaging head 110 that provides visualization of the extended tool system 108 and the target tissue 104. The imaging head 110 may include an imaging device 111 that captures images. In use, the imaging head 110 may be placed against or close to the wall of the passageway 102 to minimize or eliminate air between the imaging head and tissue. The imaging head 110 may include, for example, an ultrasound or optical imaging array for capturing images in an imaging field of view 112. The imaging head 110 may include a plurality (e.g., one or more arrays) of imaging elements. The imaging elements may be arranged to be any size, configuration, or shape. For example, the imaging elements may be arranged along a surface to form an array having a moon, ring, circle, or rectangle pattern. In some embodiments, the imaging elements include an array of transducers (e.g., lead zirconate titanate (PZT) transducers) that generate ultrasound waves and/or detect reflected ultrasound waves. In some embodiments, the imaging elements include an array of ultrasound receivers (e.g., whisper gallery mode (WGM) resonators) and an array of ultrasound transmitters (e.g., piezoelectric array). The imaging probe 100 may be steerable to navigate the imaging probe 100 to a deployment location near the target tissue 104. For example, a plurality of pull wires or tendons (not shown) may extend along a length of the imaging probe 100 and may be manipulated to steer the distal end of the imaging probe. Alternatively, the imaging probe 100 may be passively flexible and may be navigated to a deployment location by a steerable catheter or sheath having a lumen through which the imaging probe is disposed. In some examples, imaging probe 100 may include a lumen for receipt of a guidewire to navigate the imaging probe to a deployment location.

The tool system 108 may include, for example, a biopsy tool (e.g. a tissue piercing tool such as a needle for tissue cutting and/or rotating coring, a cryo-biopsy tool, a forceps instrument, a brush tool) or a therapy tool (e.g., a cryo tool, a radio frequency (RF) or microwave (MW) ablation tool, an electroporation tool, a suction device). In some examples, the tool system 108 may include a (e.g., tissue piercing) tool 116 that extends through a sheath 118. The tool 116 may be shrouded by the sheath 118 when the tool 116 is within the channel 106 of the imaging probe 100 to protect the channel 106 and distal tip of the tool 116. The tool 116 may extend distally of the sheath 118 when the distal end of the tool 116 is distal of the aperture 107. Image data from the imaging device 111 may be captured to indicate whether the tool 116 has pierced or failed to engage the tissue 104. The image data may also show where the tool 116 has pierced the tissue 104, whether the tool 116 is within a threshold distance from vulnerable tissue (such as vasculature or pleural tissue), or other interactions between the tool 116 and anatomic structures within the field of view 112.

In the example of FIG. 1, the shaft 109 of the imaging probe 100 may generally extend along a longitudinal axis A1 with the channel 106 also extending generally parallel to the axis A1. The aperture 107 may be near the base of the imaging head 110. As compared to examples in which the channel has a distal curved portion which the width of the imaging probe must accommodate, the channel 106 extending parallel to the axis A1 may allow the imaging probe to be constructed with a reduced diameter. In some examples, the shaft 109 of the imaging probe 100 may have an outer width between approximately 3.0 and 20.0 mm, and the tool 116 may have an outer width between approximately 1.0 and 3.5 mm.

In some examples, the imaging head 110 may be angled away from the longitudinal axis A1 by an angle 114. If the tool system 108 extends from the aperture 107 in a direction generally parallel to the longitudinal axis A1 or at an angle that is too close or too far from the imaging head 110, the tool 116 may not be visible within the field of view 112 of the imaging device 111. As described in the examples herein, the tool 116 and/or the imaging probe 100 may be configured such that the tool 116 predictably and repeatably extends into the field of view 112 or a particular or predetermined portion of the field of view 112 when deployed from the channel 106.

In some examples, a distal portion 117 of the tool 116 may be biased to form a bend at a bend portion 120 In one example, the bias may result from the bend portion 120 having a bent shape. A “bent shape,” as used herein, refers to a curvature of the bend portion 120 in a rest state where no external force is applied to the tool 116. The tool 116 may be flexible such that the bent shape may be changed (e.g., straightened or bent at other angles) when forces are applied to the tool 116. In another example, the bias may result from a flexible tool having a straight shape being passed through a curved channel portion of the camera probe. The bend portion 120 may bend in a single direction, such as about the axis Zₚ or in a single plane, such as plane XₚYₚ to ensure that the tool 116 is visible in the plane of the field of view 112.

In some examples, the distal portion 117 may be the length of the tool 116 distal of and inclusive of the bend portion 120. In some examples, the bend portion 120 may include all or most of the distal portion 117, such as occurs if the distal portion forms a continuous arc. In some examples, a section of the distal portion 117 distal of the bend portion 120 may be relatively straight. In some examples, the distal portion 117 of tool 116 may be biased to form a bend angle 122 between the imaging head 110 and the tool 116. The bend angle 122 may comprise the angle 114 between the imaging head 110 and the axis A1 and an angle 123 between the axis A1 and the tool 116. In some examples, the angle 114 may be approximately 35 degrees, and the angle 123 may be between approximately 30 and 45 degrees. In some examples, the bend angle 122 may be between approximately 20 and 80 degrees. The bend portion 120 may cause at least a section or a distal tip 119 of the distal portion 117 of the tool 116 to extend into the field of view 112. The bend portion 120 may also allow the tool 116 to resist bending outside of the plane of the predetermined bend.

In some examples, the bend portion 120 or the entire distal portion 117 may be formed of a flexible, shape memory material, such as nitinol, that allows the portion 120 to be straightened while the distal portion 117 extends within the channel 106 (e.g., where interior wall of the channel 106 applies forces on the portion 120) and to form a bent shape when the distal portion is extended outside of the channel 106 and released from the constraint of the channel 106. The shape memory material forming the bend portion 120 may predictably and repeatedly generate the same bend angle 122 between the distal portion 117 of the tool 116 and the imaging head 110. In some examples, the shape of the bend portion 120 may be heat set during manufacturing to form the bent shape. In some examples, the tool 116 may include a channel into which a bend member such as a stylet may be inserted. The stylet may have a preformed bend and may be formed of a flexible, shape memory material, such as nitinol. A nitinol stylet, for example, may be shape set with a specific curve. The stylet may guide the tool in the appropriate curved path and may be removed when the tool is used such as for drawing a tissue sample through the needle. The stylet may also have an oval, elliptical or rectangular profile that is more flexible in a first bending plane than in a second perpendicular bending plane.

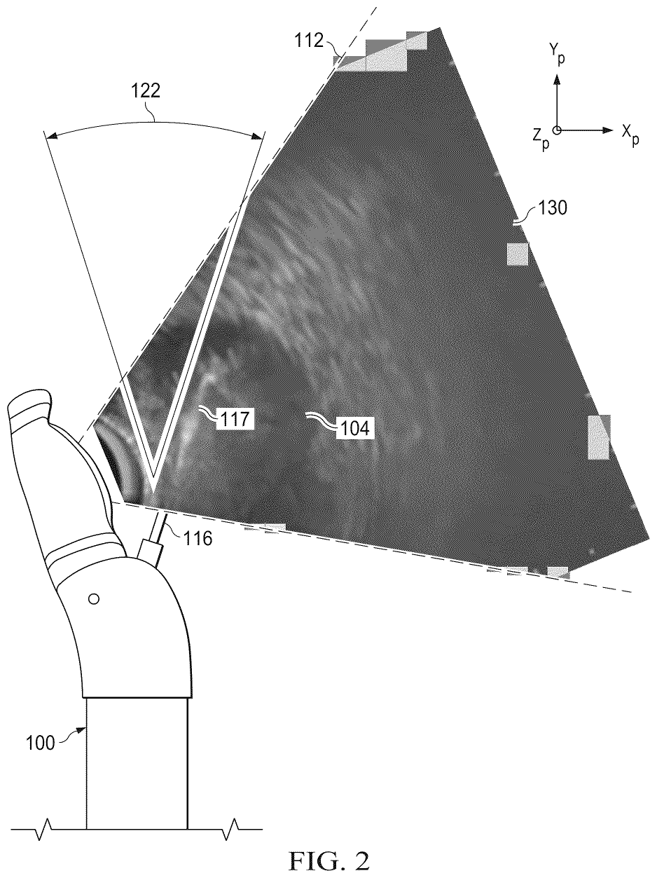

FIG. 2 illustrates an image 130 of the field of view 112 relative to the imaging probe 100 that captured the image 130. In this example, the field of view 112 may have a fan-shape that is smaller or narrower near the tool 116 and larger or wider farther from the tool 116. In other words, the field of view may increase as distance from the imaging device increases. In this example, the plane of the image 130 may generally correspond to the X_{P}Y_{P} plane of the imaging probe. The image 130 of the field of view 112 may include the target tissue 104 and the distal tip 119 or a section of the distal portion 117 of the tool 116. The bend angle 122 causes the tool 116 to extend within the field of view 112 to be visualized within the image 130. In particular, the field of view 112 may expand as distance from imaging probe 100 increases. If the tool 116 does not bend away from the imaging probe 100, then the tool 116 may be at a region where the field of view 112 is small or narrow and may not be sufficiently visible within the field of view 112. If the tool 116 bends away from the image probe 100, the tool may be located at more distant region where the field of view 112 is larger or broader, and thus is more fully visible within the field of view 112. Further, image resolution directly proximate the imaging probe 100 may be low due, for example, to the ring down associated with the transducer. Consequently, placing the tool away from the imaging probe may provide a better image. Additionally, directing the tool away from the imaging probe may allow the tool to extend deeper into the tissue beyond the anatomic passageway wall (e.g. in a direction transverse to the wall) to access more distal tissues. The image 130 may show, for example, whether the tool 116 has pierced the target tissue 104 and may further illustrate where the piercing occurred, the depth of the piercing, and/or whether any nearby vulnerable tissues are in jeopardy. Additionally, information about the location of the tool, such as a biopsy tool, may be useful for sampling at a central necrosis and at margins or edges of a lymph node. These types of tissue may be echogenic and may contain cancerous tissue. If the tool 116 is a biopsy tool, for example, visualizing the tool 116 in the image 130 relative to a lesion may allow a clinician to observe whether the lesion was engaged by the biopsy tool and decide whether to engage other areas of the lesions to perform additional biopsies. In some examples, the image 130 may be displayed (e.g., on a display system 1110) to a clinician or other user. In some examples, the image data may be analyzed by image analysis software to record various information such as the location of the piercing, the depth of the piercing, and/or proximity to vulnerable tissues.

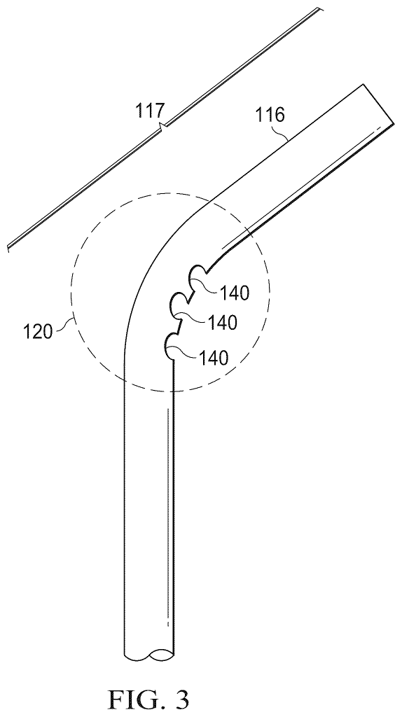

The bent shape of the tool may be achieved in various ways. In some examples, as shown in FIG. 3, the bend portion 120 may include surface features 140 at the inside radius of the bend portion 120 of the tool 116 that create regions of wall weakness, encourage the preferential bend, and reduce the bending stiffness of the tool 116 in the area of the bend. The surface features 140 may include slits (e.g., formed via laser cutting), holes, or other perforations. A sealing member (not shown) in the form of a flexible tube or sleeve (e.g., a polyethylene terephthalate (PET) sleeve) may extend across the perforations to allow a vacuum force to evacuate material through the tool and to prevent migration of fluid or material through the perforations.

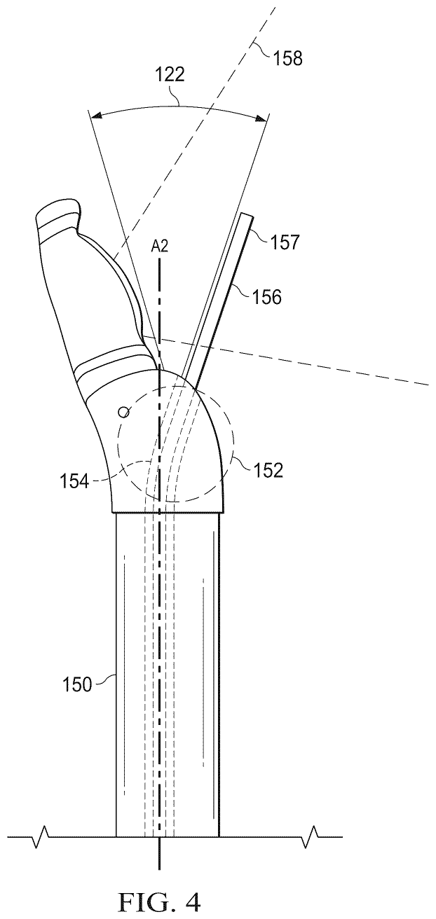

In some examples, as shown in FIG. 4, an imaging probe 150 may include a bend section 152 generated by a curved channel portion 154 extending within the imaging probe 150. The curved channel portion 154 refers to a distal portion of the channel of the imaging probe 150 that has a curved shape to impart a bend on a flexible tool 156. The curved channel portion 154 may cause the flexible tool 156 to form the bent shape and be directed away from a longitudinal axis A2 at the bend section 152 so that a distal portion 157 of the tool 156 extends distally from the channel 154 at the bend angle 122 and into a field of view 158. The curved channel portion 154 allows a flexible tool 156 having an unbent shape to conform to the bend of the curved channel 154. Optionally, the flexible tool 156 may also include a preformed bend, as described for FIG. 1, to contribute to the bend portion and the formation of the bend angle. In other aspects, the imaging probe 150 may be similar to the probe 100, and the tool 156 may be similar to the tool 116. In some examples, if the tool has a bent shape, as described for FIG. 1, the probe may not additionally include a curved channel portion to form a bend in the tool. Thus, if the tool has a bent shape, the imaging probe may have a smaller diameter because it may include a straight channel or straighter channel that omits the curved channel portion.

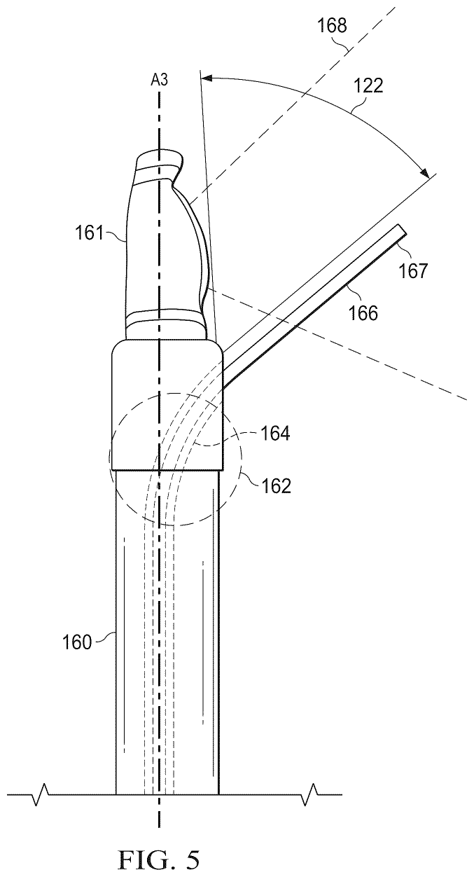

The imaging head of the imaging probe may be bent or straight with respect to the longitudinal axis of the imaging probe. In some examples, as shown in FIG. 5, an imaging probe 160 may include an imaging head 161 that is generally aligned with (e.g., at a 0 degrees angle with respect to) a longitudinal axis A3 of the probe 160. The imaging probe 160 may include a predetermined bend section 162 generated by a curved channel portion 164 extending within the imaging probe 160. The curved channel portion 164 may cause a flexible tool 166 extending therethrough to bend away from a longitudinal axis A3 at the bend section 162 so that the distal portion 167 of the tool 166 extends distally from the channel 164 at the predetermined bend angle 122 and into a field of view 168. As compared to the example of FIG. 4, the bend of the tool 166 relative to axis A3 may be larger so that the tool 166 extends into the field of view of the imaging head. The bend of the tool 156 (FIG. 4) relative to axis A2 may be smaller than the bend of the tool 166 because the imaging head is angled in the opposite direction of the bend. The angled imaging head (with respect to axis A2) as in FIG. 4 may allow for a gentler or smaller bend (with respect to axis A2) in the tool (as compared to the example of FIG. 5) while preserving a bend angle 122 that places the tool within the field of view of the imaging probe. In the example of FIG. 4, the curved channel portion may have a larger radius of curvature (as compared to FIG. 5) because the channel does not need to impart as much curvature on the tool. A straight imaging head may, however, more easily traverse small anatomical passageways. In examples where the tool itself is formed with a bent shape (e.g., tool 116), the shape bias provides the curvature, not a curved channel portion and thus a bent tool may also be used with the straight imaging head to minimize the imaging probe diameter. The angle of the imaging head 161 and the curved channel portion are independent features. For example, either a bent or unbent imaging head may be used with a straight channel. Optionally, the flexible tool may also include a preformed bend, as described for FIG. 3, to contribute to the bend portion and the formation of the bend angle. In other aspects, the imaging probe 160 may be similar to the probe 100, and the tool 166 may be similar to the tool 116.

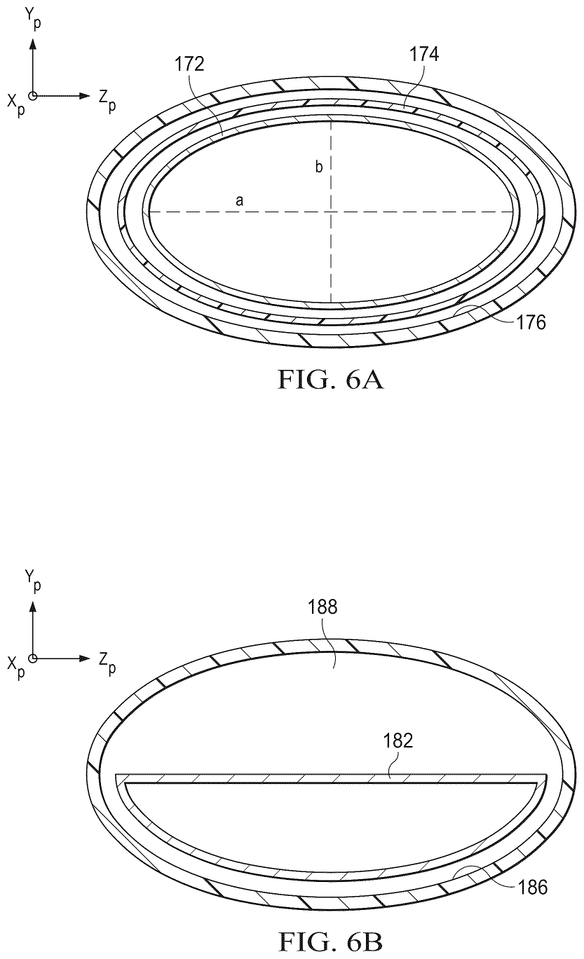

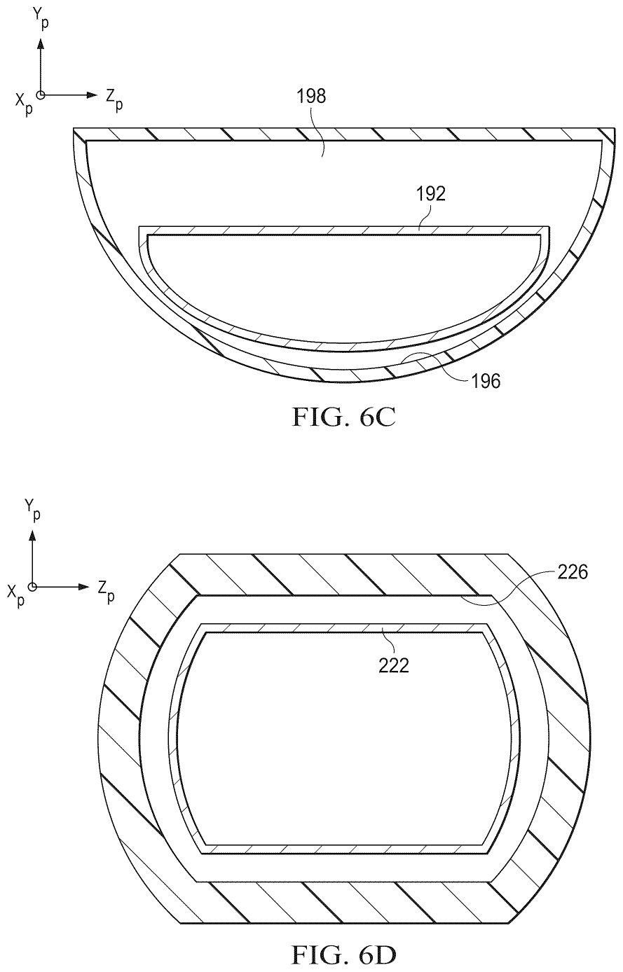

The tool may be rotationally constrained in various ways to ensure bending away from the imaging probe and into the field of view of the imaging probe and to prevent undesirable bending outside of a plane of the field of view (e.g., the X_{P}Y_{P} plane). In some examples, as shown in FIGS. 6A, 6B, 6C, and 7, the cross sectional shape of the tool and/or channel of the imaging probe may facilitate rotational locking of the tool with respect to respect to the imaging probe and facilitate bending of the tool along in a desired plane (e.g., corresponding with the imaging plane of the imaging probe). The tool and/or channel of the imaging probe may include cross-sectional shapes that are asymmetrical (e.g., different dimensions) in orthogonal directions. For example, an asymmetrical cross-sectional shape may include a major dimension that is wider than a minor dimension defined orthogonal to the major dimension. The tool may bend along the minor dimension, either based on having a bent shape or having a straight shape that is bent along the minor dimension by a curved channel portion of the imaging probe. The asymmetrical cross-sectional shape may result in the tool being resistant to bending along the major dimension and may facilitate bending along (e.g., only) the minor dimension. In some embodiments, the channel of the imaging probe may have an asymmetrical cross-sectional shape that constrains the rotational orientation of the tool within the channel. These rotational constraints may help to ensure that the tool (e.g., a tool with a shape memory bend) will extend from the imaging probe in a desired direction and prevent undesirable bending in other directions.

FIG. 6A illustrates a cross-sectional view of a channel 176 of an imaging probe and a tool 172 along a cross-sectional plane, such as plane 124 of FIG. 1. All or a portion of the length of tool 172 may be shrouded by a sheath 174 when the tool 172 is within the channel 176 of the imaging probe. In some examples, the tool 172 may be similar to the tool 116, except as described. For example, the tool 172 may be a tissue piercing tool or some other type of tool. In this example, tool 172 may have a cross-sectional shape in the form of an ellipse sized to fit within an ellipse-shaped channel 176. The corresponding shapes of the tool 172 and the channel 176 may cooperate to rotationally constrain the tool to prevent rotation or twisting of the tool 172 within the channel 176 and when the tool is extended outside of the channel 176. For example, the cross-sectional shape of the tool has a wider dimension along the Z_{P} axis and a narrower dimension along the Y_{P} axis. Furthermore, the tool 172 may have a bent shape that includes curvature in the X_{P}Y_{P} plane. This causes the tool 172 to resist bending in the X_{P}Z_{P} plane and facilitates bending only in the X_{P}Y_{P} plane (e.g., which is aligned to the imaging plane of the imaging probe). In other words, the tool 172 may have a moment of inertia about a minor dimension b that is greater than a moment of inertia about a major dimension a. For example, the moment of inertia I_{Y}=[π(b/2)(a/2)³]/4 may greater than the moment of inertia I_{Z}=[π(a/2)(b/2)³]/4. The cross sectional shape of the tool and/or channel may have various other shapes that provide such rotational constraints. In some examples, the cross-sectional shapes of the tool and/or channel may be oblong, oval, or other symmetrical or asymmetrical shapes with unequal moments of inertia about the orthogonal axes.

In some examples, a fluid, such as air or an irrigation fluid, may be permitted to flow through the channel 176 around the sheath 174. In one example, saline may be injected to clean debris in the anatomic area to improve visibility if an imaging system, such as a camera, is incorporated in the tool or delivery catheter. Saline or other fluid may also aid in ultrasound coupling between the ultrasound probe and the passageway wall.

FIG. 6B illustrates a cross-sectional view of a channel 186 of an imaging probe and a tool 182 along a cross-sectional plane, such as plane 124 of FIG. 1. All or a portion of a length of the tool 182 may be shrouded by a sheath (not shown) when the tool 182 is within the channel 186 of the imaging probe. In some examples, the tool 182 may be similar to the tool 116, except as described. For example, the tool 182 may be a tissue piercing tool or some other type of tool. In this example, tool 182 may have a cross-sectional D-shape that is sized to fit within an ellipse-shaped channel 186. The shapes of the tool 182 and the channel 186 may cooperate to constrain the tool to prevent rotation or twisting within the channel 186 and when the tool is extended outside of the channel 186. For example, the cross-sectional shape of the tool has a wider dimension along the Z_{P} axis and a narrower dimension along the Y_{P} axis. Furthermore, the tool 182 may have a bent shape that includes curvature in the X_{P}Y_{P} plane. This causes the tool 182 to resist bending in the X_{P}Z_{P} plane and facilitates bending only in the X_{P}Y_{P} plane (e.g., which is aligned to the imaging plane of the imaging probe). In this example, a fluid passageway 188 may extend between a flat side of the tool 182 and the channel 186. The passageway 188 may be formed by a flexible conduit or may be the remainder of the open space not occupied by the tool 182. Generally, a fluid passageway may extend between the channel of the probe and the tool along a length over which the cross-sectional shape of the channel is different from the cross-sectional shape of the tool.

FIG. 6C illustrates a cross-sectional view of a channel 196 and of a tool 192 along a cross-sectional plane, such as plane 124 of FIG. 1. All or a portion of a length of the tool 192 may be shrouded by a sheath (not shown) when the tool 192 is within the channel 196 of the imaging probe. In some examples, the tool 192 may be similar to the tool 116, except as described. For example, the tool 192 may be a tissue piercing tool or some other type of tool. For example, the cross-sectional shape of the tool has a wider dimension along the Z_{P} axis and a narrower dimension along the Y_{P} axis. Furthermore, the tool 192 may have a bent shape that includes curvature in the X_{P}Y_{P} plane. This causes the tool 192 to resist bending in the X_{P}Z_{P} plane and facilitates bending only in the X_{P}Y_{P} plane (e.g., which is aligned to the imaging plane of the imaging probe). In this example, tool 192 may have a cross-sectional D-shape that is sized to fit within a larger D-shaped channel 196. The shapes of the tool 192 and the channel 196 may cooperate to constrain the tool to prevent rotation or twisting within the channel 196 and when the tool is extended outside of the channel 196. In this example, a fluid passageway 198 may extend through the channel 196. The passageway 198 may be formed by a flexible conduit or may be the remainder of the open space not occupied by the tool 192. As compared to the examples of FIG. 6B, the example of FIG. 6C may allow for a decreased dimension of the channel 196 in the X_{P}Y_{P} direction.

FIG. 6D illustrates a cross-sectional view of a channel 226 and of a tool 222 along a cross-sectional plane, such as plane 124 of FIG. 1. All or a portion of a length of the tool 222 may be shrouded by a sheath (not shown) when the tool 222 is within the channel 226 of the imaging probe. In some examples, the tool 222 may be similar to the tool 116, except as described. For example, the tool 222 may be a tissue piercing tool or some other type of tool. For example, the cross-sectional shape of the tool has a wider dimension along the Z_{P} axis and a narrower dimension along the Y_{P} axis. Furthermore, the tool 222 may have a bent shape that includes curvature in the X_{P}Y_{P} plane. This causes the tool 222 to resist bending in the X_{P}Z_{P} plane and facilitates bending only in the X_{P}Y_{P} plane (e.g., which is aligned to the imaging plane of the imaging probe). In this example, tool 222 may have a bowed-rectangle shape that is sized to fit within a bowed-rectangle shaped channel 226. The shapes of the tool 222 and the channel 226 may cooperate to constrain the tool to prevent rotation or twisting within the channel 226 and when the tool is extended outside of the channel 226. In this example, a shaped mandrel may be used to form the shape 226. For example, a metal mandrel shaped may be formed from a round rod with two machined flats to achieve the shape as shown. In some examples, the tool 222 may be manufactured into an oval by first starting with a round needle and flattening into an oval profile with a press.

FIG. 6E illustrates a cross-sectional view of a channel 236 and of a tool 232 along a cross-sectional plane, such as plane 124 of FIG. 1. All or a portion of a length of the tool 232 may be shrouded by a sheath (not shown) when the tool 232 is within the channel 236 of the imaging probe. In some examples, the tool 232 may be similar to the tool 116, except as described. For example, the tool 232 may be a tissue piercing tool or some other type of tool. For example, the cross-sectional shape of the tool has a wider dimension along the Z_{P} axis and a narrower dimension along the Y_{P} axis. Furthermore, the tool 232 may have a bent shape that includes curvature in the X_{P}Y_{P} plane. This causes the tool 232 to resist bending in the X_{P}Z_{P} plane and facilitates bending only in the X_{P}Y_{P} plane (e.g., which is aligned to the imaging plane of the imaging probe). In this example, tool 232 may have an oval or racetrack shape that is sized to fit within an oval or racetrack shaped channel 236. The shapes of the tool 232 and the channel 236 may cooperate to constrain the tool to prevent rotation or twisting within the channel 236 and when the tool is extended outside of the channel 236. In this example, a shaped mandrel may be used to form the shape 236. In some examples, the tool 232 may be manufactured into an oval by first starting with a round needle and flattening into an oval profile with a press.

FIG. 7 illustrates a cross-sectional view of a channel 206 and of a tool 202 along a cross-sectional plane, such as plane 124 of FIG. 1. All or a portion of the tool 202 may be shrouded by a sheath (not shown) when the tool 202 is within the channel 206 of the imaging probe. In some examples, the tool 202 may be similar to the tool 116, except as described. For example, the tool 202 may be a tissue piercing tool or some other type of tool. In this example, tool 202 may have an egg-shape that is sized to fit within a circular channel 206. Guides 207 such as protrusions, rails, or troughs, may constrain rotational motion of the tool 202. The constraints may cause the tool 202 to resist bending in the X_{P}Z_{P} plane and facilitate bending only in the X_{P}Y_{P} plane (e.g., which is aligned to the imaging plane of the imaging probe). In this example, fluid passageways 208 may extend on the sides of the tool 202. The passageways 208 may be formed by a flexible conduit or may be the remainder of the open space not occupied by the tool 202.

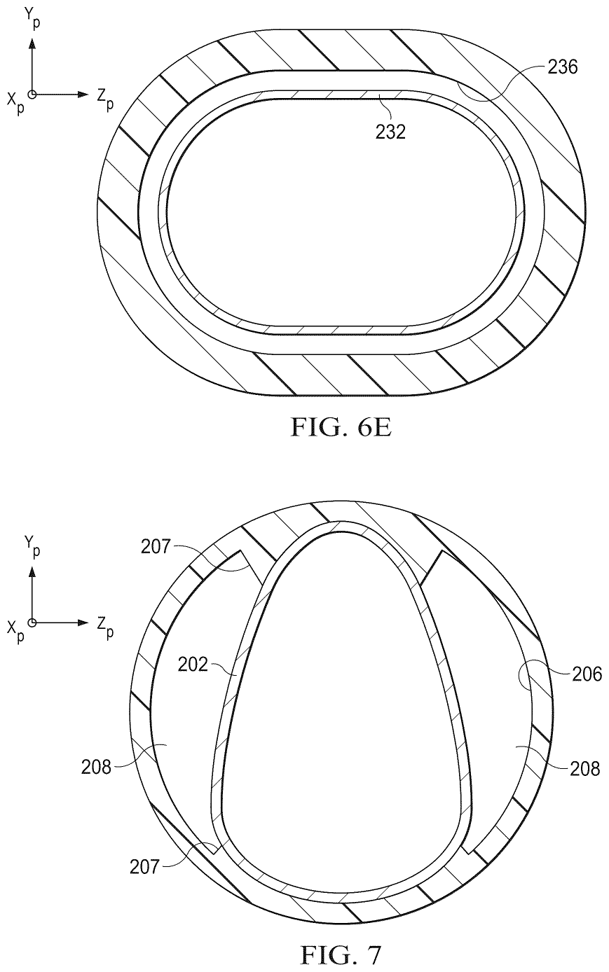

FIG. 8A illustrates an imaging probe 250 with a tool 252 rotationally constrained relative to a working channel 254 of the probe. The imaging probe 250 may include a section 256 having a bend caused by a curved channel portion 258 extending withing the imaging probe 250. The curved channel portion 254 may cause the flexible tool 252 extending therethrough to bend away from a longitudinal axis A4, such as at the section 256, so that the distal portion 257 of the tool 252 extends distally from the channel 254 at a desired bend angle and into a field of view. In this example, the tool 252 may be a tissue piercing tool with a beveled tip 260. The tool 252 may be rotationally constrained relative to the channel 254 (as described in any of the above examples) so that the beveled tip 260 travels along the inside radius 259 of the bend section 256 when the tool 252 is inserted or retracted in the channel 254. This reduces the likelihood or force of contact between the tip 260 of the bevel and the outside radius of the bend section 256, thus preventing damage to the tool 252 or the channel 254. In other examples, the tip 260 of the tool may be bent inward (e.g., toward the center of the tool) to prevent contact with a sheath lining the channel 254 or with the channel 254 itself.

FIG. 8B illustrates a cross-sectional view of a channel 266 and of a tool 262 along a cross-sectional plane, such as plane 124 of FIG. 1. All or a portion of a length of the tool 262 may be shrouded by a sheath (not shown) when the tool 262 is within the channel 266 of the imaging probe. In some examples, all or a portion of a length of the channel 266 may be lined by a sheath (not shown) when the tool 262 is within the channel 266 of the imaging probe. In some examples, the tool 262 may be similar to the tool 116, except as described. For example, the tool 262 may be a tissue piercing tool with a pointed tip 264 or some other type of tool. For example, the cross-sectional shape of the tool has a wider dimension along the Z_{P} axis and a narrower dimension along the Y_{P} axis. Furthermore, the tool 262 may have a bent shape that includes curvature in the X_{P}Y_{P} plane. This causes the tool 262 to resist bending in the X_{P}_{Z} P plane and facilitates bending only in the X_{P}Y_{P} plane (e.g., which is aligned to the imaging plane of the imaging probe). In this example, tool 262 may have an oval or racetrack shape that is sized to fit within a channel 266. The channel 266 may have a generally oval or racetrack shape, but a portion of the inner profile may have a recessed area 265 to accommodate the tip 264 and prevent contact between the tip 264 and the channel 266. The shapes of the tool 262 and the channel 266 may cooperate to constrain the tool to prevent rotation or twisting within the channel 266 and when the tool is extended outside of the channel 266.

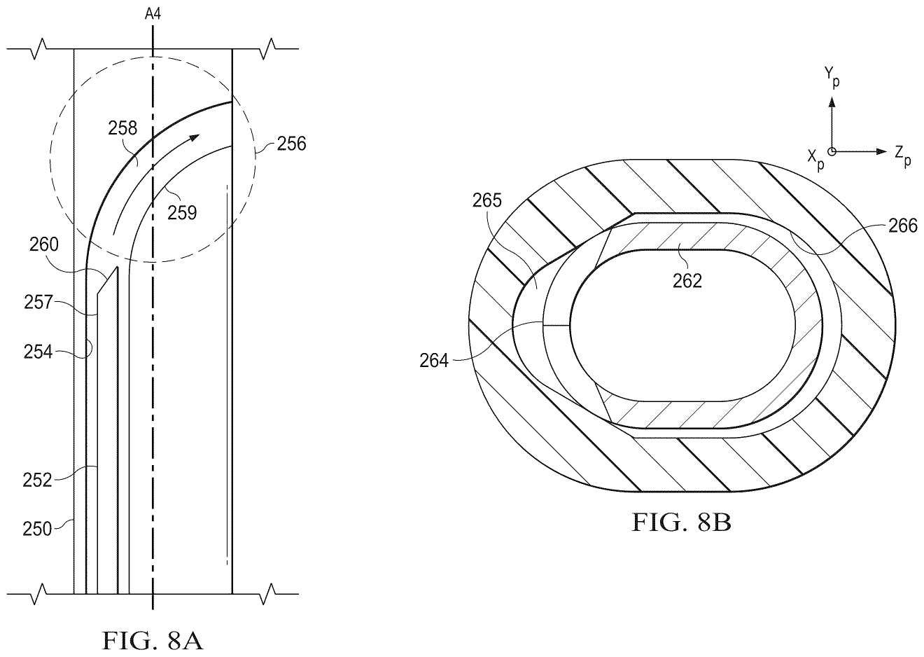

FIG. 9 illustrates a tool 300 that may be similar to tool 116 and may be used as any of the tissue piercing tools previously described. In this example, the tool 300 includes a bend portion 302 that may be formed of a flexible material (e.g., an elastomeric material) that allows the tool to extend to a straightened configuration when constrained within a working channel and return to a bent shape when released from constraint distal of the channel. A portion 304 of the tool 116 distal of the bend portion 302 may be made of a more rigid material (e.g., stainless steel or a rigid polymer). The rigid portion 304 may resist deformation when piercing tissue, as compared to a more flexible or bendable material.

In some examples, the tool may be bent to a variety of angles. A variable bias or bend in the angle of the distal end of the tool relative to the shaft of the probe may be achieved, for example, with adjustable pull wires extending through the tool. As another example, a variable bias may be achieved with a bi-metal strip in the distal portion of the tool that may be heated via electrical current resistance that causes the strip to flex. As another example, the bend in the tool may be varied using a stylet that extends within the tool with a variable curvature or multiple stylets with different curvatures. If the tool has a variable bend, a clinician or other user viewing an image of the field of view may adjust the bend in the tool to cause the tool to bend into the plane of the field of view.

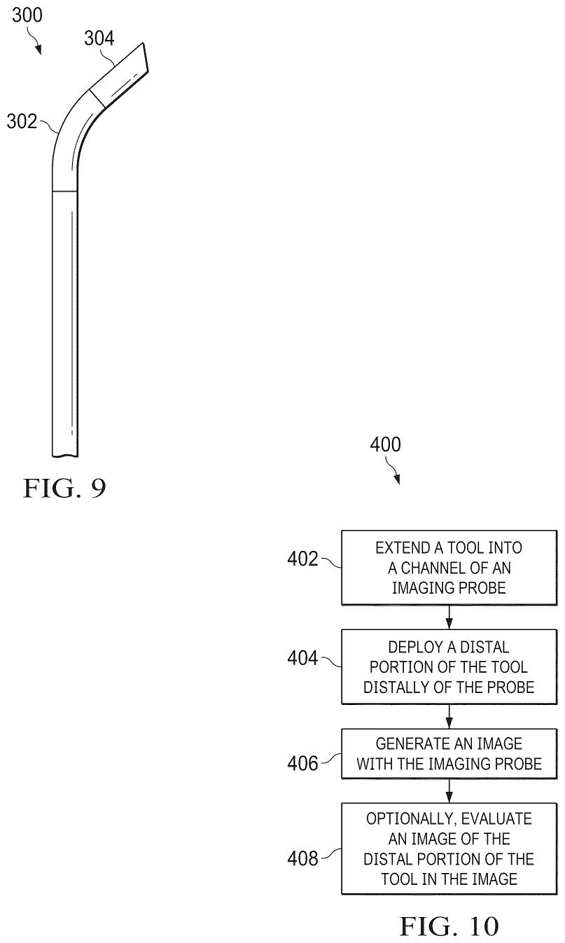

FIG. 10 provides a flowchart illustrating a method 400 for performing a medical imaging procedure using an imaging probe and a tool, such as a tissue piercing tool. The method 400 is illustrated as a set of operations or processes that may be performed in the same or in a different order than the order shown. One or more of the illustrated processes may be omitted in some examples of the method. Additionally, one or more processes that are not expressly illustrated in FIG. 10 may be included before, after, in between, or as part of the illustrated processes. In some examples, one or more of the processes of method 400 may be implemented, at least in part, by a control system executing code stored on non-transitory, tangible, machine-readable media that when run by one or more processors (e.g., the processors of a control system) may cause the one or more processors to perform one or more of the processes.

Before or after navigating an imaging probe (e.g., imaging probe 100) to a deployment location in the vicinity of a target tissue (e.g., target tissue 104), a tool (e.g. a tool 116) may be extended into a channel of the imaging probe, at a process 402. At a process 404, a distal portion of the tool may be deployed from the imaging probe. The distal portion of the tool may include a bent shape that causes the distal portion of the tool to bend into a field of view of the imaging probe. In some examples, the tool may be constrained by (e.g., asymmetrical) shapes of the channel and tool to resist bending outside of the plane of the field of view of the imaging probe. This allows all or substantially all the distal portion of the tool to be captured in the field of view. At a process 406, an image may be generated by the imaging probe while the tool is extended distally from the probe. In some examples, images may be continuously generated by the imaging probe throughout the method 400, including during the processes 402, 404, and 408. Consequently, images may be generated before the tool is deployed, as the tool is being deployed, while the tool is an operational position, as the tool is being retracted into the probe, and after the tool is withdrawn from the imaging probe. At an optional process 408, an evaluation may be performed using image analysis techniques or clinician observation. The evaluation may include a determination of whether the tool is visible in the field of view of the probe, whether the tool has pierced a target tissue, the depth the tool has extended into the target tissue, whether the location of the tool may or has jeopardized vulnerable tissues.

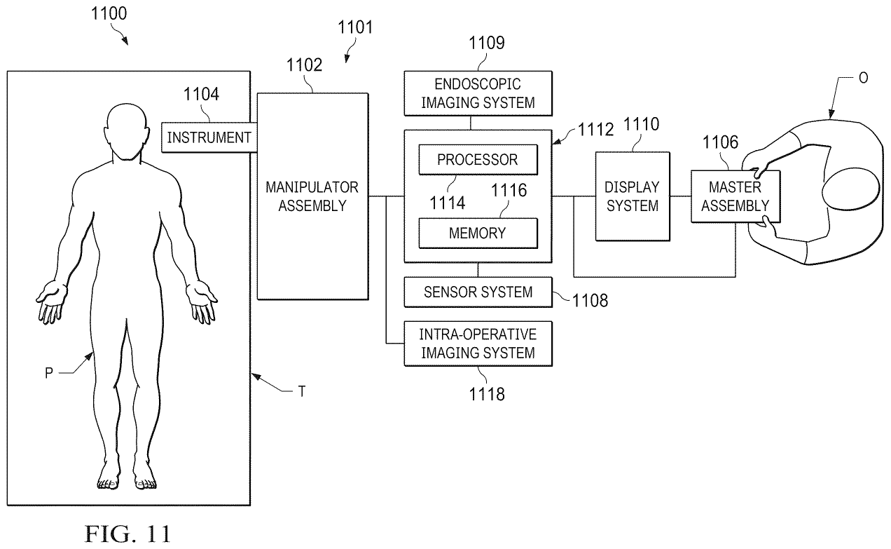

In some examples, medical procedure may be performed using hand-held or otherwise manually controlled imaging probes and tools of this disclosure. In other examples, the described imaging probes and tools many be manipulated with a robot-assisted medical system as shown in FIG. 11. FIG. 11 illustrates a robot-assisted medical system 1100. The robot-assisted medical system 1100 generally includes a manipulator assembly 1102 for operating a medical instrument system 1104 (including, for example, imaging probe 100 and tool system 108) in performing various procedures on a patient P positioned on a table T in a surgical environment 1101. The manipulator assembly 1102 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. A master assembly 1106, which may be inside or outside of the surgical environment 1101, generally includes one or more control devices for controlling manipulator assembly 1102. Manipulator assembly 1102 supports medical instrument system 1104 and may include a plurality of actuators or motors that drive inputs on medical instrument system 1104 in response to commands from a control system 1112. The actuators may include drive systems that when coupled to medical instrument system 1104 may advance medical instrument system 1104 into a naturally or surgically created anatomic orifice. Other drive systems may move the distal end of medical instrument system 1104 in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulatable end effector of medical instrument system 1104 for grasping tissue in the jaws of a biopsy device and/or the like.

Robot-assisted medical system 1100 also includes a display system 1110 (which may display image 130 of FIG. 2) for displaying an image or representation of the surgical site and medical instrument system 1104 generated by a sensor system 1108 and/or an endoscopic imaging system 1109. Display system 1110 and master assembly 1106 may be oriented so operator O can control medical instrument system 1104 and master assembly 1106 with the perception of telepresence.

In some examples, medical instrument system 1104 may include components for use in surgery, biopsy, ablation, illumination, irrigation, or suction. Medical instrument system 1104, together with sensor system 1108 may be used to gather (i.e., measure) a set of data points corresponding to locations within anatomic passageways of a patient, such as patient P. In some examples, medical instrument system 1104 may include components of the endoscopic imaging system 1109, which may include an imaging scope assembly or imaging instrument (such as imaging probe 100) that records a concurrent or real-time image of a surgical site and provides the image to the operator or operator O through the display system 1110. The concurrent image may be, for example, a two or three-dimensional image captured by an imaging instrument positioned within the surgical site. In some examples, the endoscopic imaging system components may be integrally or removably coupled to medical instrument system 1104. However, in some examples, a separate endoscope, attached to a separate manipulator assembly may be used with medical instrument system 1104 to image the surgical site. The endoscopic imaging system 1109 may be implemented as hardware, firmware, software, or a combination thereof which interact with or are otherwise executed by one or more computer processors, which may include the processors of the control system 1112.

The sensor system 1108 may include a position/location sensor system (e.g., an electromagnetic (EM) sensor system) and/or a shape sensor system for determining the position, orientation, speed, velocity, pose, and/or shape of the medical instrument system 1104.

Robot-assisted medical system 1100 may also include control system 1112. Control system 1112 includes at least one memory 1116 and at least one computer processor 1114 for effecting control between medical instrument system 1104, master assembly 1106, sensor system 1108, endoscopic imaging system 1109, intra-operative imaging system 1118, and display system 1110. Control system 1112 (which may include a controller in operative communication with the imaging device 111) also includes programmed instructions (e.g., a non-transitory machine-readable medium storing the instructions) to implement some or all of the methods described in accordance with aspects disclosed herein, including instructions for providing information to display system 1110.

Control system 1112 may further include a virtual visualization system to provide navigation assistance to operator O when controlling medical instrument system 1104 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

An intra-operative imaging system 1118 may be arranged in the surgical environment 1101 near the patient P to obtain images of the anatomy of the patient P during a medical procedure. The intra-operative imaging system 1118 may provide real-time or near real-time images of the patient P. In some examples, the intra-operative imaging system 1118 may comprise an ultrasound imaging system for generating two-dimensional and/or three-dimensional images. For example, the intra-operative imaging system 1118 may be at least partially incorporated into an imaging probe such as imaging probe 100. In this regard, the intra-operative imaging system 1118 may be partially or fully incorporated into the medical instrument system 1104.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 1112) or may be implemented, at least in part, in the form of executable code stored on non-transitory, tangible, machine-readable media that when run by one or more processors (e.g., the processors 1114 of control system 1112) may cause the one or more processors to perform one or more of the processes.

Any described “imaging device” herein may include an ultrasound array, optical imaging device, or any other suitable imaging hardware. Any described “imaging probe” may include an ultrasound probe, an optical imaging probe, or a probe incorporating any other suitable imaging modality. Additionally, any “ultrasound array,” “imaging array,” or “imaging device” as described herein may comprise a single imaging element (e.g., transducer) or a plurality of such devices.

One or more elements in examples of this disclosure may be implemented in software to execute on a processor of a computer system such as control processing system. When implemented in software, the elements of the examples of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In one example, the control system supports wireless communication protocols such as Bluetooth, IrDA, HomeRF, IEEE 802.11, DECT, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom-e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

While certain exemplary examples of the invention have been described and shown in the accompanying drawings, it is to be understood that such examples are merely illustrative of and not restrictive on the broad invention, and that the examples of the invention not be limited to the specific constructions and arrangements shown and described, since various other modifications may occur to those ordinarily skilled in the art.

1. An apparatus comprising:
an imaging probe including:
a channel extending through the imaging probe and terminating at an opening; and
an imaging device configured to generate image data having a field of view; and
a tool configured to slidably extend within the channel, wherein a portion of the tool is flexible, has a bent shape, and bends away from the imaging device when the portion of the tool is extended distally of the opening.

2. The apparatus of claim 1, wherein the portion of the tool bends in a direction that results in a distal tip of the tool being within the field of view of the imaging device.

3. The apparatus of claim 1, wherein the portion of the tool resists bending in a direction that results in a distal tip of the tool being outside of an imaging plane of the field of view.

4. The apparatus of claim 1, wherein the field of view increases as distance from the imaging device increases.

5. (canceled)

6. The apparatus of claim 1, wherein the bent shape of the tool is formed of a flexible, shape memory material.

7. The apparatus of claim 1, wherein the bent shape of the tool is formed by surface features on the portion of the tool.

8. (canceled)

9. The apparatus of claim 1, wherein the tool is constrained to prevent rotation of the tool within the channel.

10. The apparatus of claim 9, wherein each of the tool and the channel has a cross-sectional shape that is asymmetrical in orthogonal dimensions.

11. The apparatus of claim 10, wherein a cross-sectional shape of the channel is oval and a cross-sectional shape of the tool is oval.

12. The apparatus of claim 10, wherein a cross-sectional shape of the channel is oval and a cross-sectional shape of the tool is D-shaped.

13. The apparatus of claim 10, wherein a cross-sectional shape of the channel is D-shaped and a cross-sectional shape of the tool is D-shaped.

14. The apparatus of claim 1, wherein a cross-sectional shape of the channel is different from a cross-sectional shape of the tool.

15. The apparatus of claim 1, wherein the imaging device is bent away from a longitudinal axis of the channel in a first direction and the portion of the tool is bent away from the longitudinal axis of the channel in a second direction opposite to the first direction.

16. The apparatus of claim 15 wherein a bend angle between the imaging device and the tool is between 20 and 80 degrees.

17. The apparatus of claim 1 wherein the tool is a tissue piercing tool including a beveled tip and the tool is rotationally constrained relative to the channel.

18. The apparatus of claim 1, wherein the imaging probe has an outer width between approximately 3.0 and 20mm mm and the tool has an outer width between approximately 1.0 and 3.5 mm.

19. A system comprising:
an imaging probe including a channel extending through the imaging probe and terminating at an opening and an imaging device configured to generate image data having a field of view; and
a tool configured to slidably extend within the channel, wherein a portion of the tool is flexible, has a bent shape, and bends away from the imaging device when the portion of the tool is extended distally of the opening; and
a control system comprising one or more processors configured to:
deploy the portion of the tool distally of the opening and into the field of view; and
receive the image data including the portion of the tool in the field of view.

20. The system of claim 19 wherein the control system is further configured to evaluate the image data to determine a position of the tool with respect to a target tissue in the field of view.

21. The system of claim 19 wherein the control system is further configured to evaluate the image data to determine a position of the tool with respect to a vulnerable tissue in the field of view.

22. The system of claim 19, wherein deploying the portion of the tool includes causing the tool to bend in a direction that results in a distal tip of the tool being within the field of view.

23-38. (canceled)
